(19) United States
(12) Patent Application Publication
(10) Pub. No.: US 2015/0230767 A1
(43) Pub. Date: Aug. 20, 2015

(54) FLAT PANEL X-RAY IMAGING DEVICE

(71) Applicant: SCANFLEX HEALTHCARE AB, Stockholm (SE)

(72) Inventors: Hans Sjöström, Jarfalla (SE); Lars Johan Johnson, Djursholm (SE)

(21) Appl. No.: 14/426,215

(22) PCT Filed: Sep. 5, 2013
(86) PCT No.: PCT/EP2013/068397

§ 371 Date: Mar. 5, 2015

(30) Foreign Application Priority Data
Sep. 5, 2012 (SE) 1250993-1

(51) Int. Cl. A61B 6/00

(52) U.S. Cl. CPC A61B 6/4441; A61B 6/4405; A61B 6/485

(57) ABSTRACT

According to embodiments, there is provided a mobile digital fluoroscopy system, comprising a mobile unit (1) having: a stand having a G-arm (18) suspended on a chassis frame (7); a first X-ray device (19) mounted on the G-arm (18) to generate X-ray images in a first plane (P1), the first X-ray device (19) having a first receiver (22) mounted on the G-arm (18) and a first transmitter (21) mounted on the G-arm (18) opposite said first receiver (22); a second X-ray device (20) mounted on the G-arm (18) to generate X-ray images in a second plane (P2) intersecting the first plane (P1) of the first X-ray device, the second X-ray device (20) having a second receiver (24) mounted on the G-arm (18) and a second transmitter (23) mounted on the arm (18) opposite said second receiver (24); wherein said first and second receivers (22) and (24) are flat digital X-ray detectors mounted at respective ends of the G-arm.

### FIELD OF THE INVENTION

The present invention relates in general to a preferably mobile digital fluoroscopy system for medical applications operating with an X-ray device mounted to generate X-ray images. More specifically, the present invention relates to a fluoroscopy system having an X-ray device provided with a flat digital X-ray detector.

### BACKGROUND

In orthopedic surgery environment, there is a need for allowing full access to the operating area with total control at each step. Therefore, X-ray imaging using C-stands or G-stands comprising imaging systems is commonly used, wherein a C-stand comprises one X-ray imaging system while a G-stand comprises two such imaging systems.

A symmetrical G-stand is generally preferable to a C-stand, since it comprises two perpendicularly mounted X-ray imaging systems, and is thereby able to provide both frontal and lateral X-ray imaging with fixed settings. The ability to simultaneously see the surgical area in both a frontal and lateral view reduces the need to move and adjust the equipment during surgery, thus reducing both surgery time and radiation dose. When the need to move the equipment is reduced, better sterility is also achieved.

The ability in a G-stand to double the surgeon's view also results in accurate positioning of implants, creating a safer and more reliable method of surgery. The angular position of the X-ray imaging systems are adjustable in relation to a patient during operation with maintained fixed relation between the intersecting planes of the generated X-ray images due to the fixed setting of the X-ray devices on the G-stand.

### RELATED ART

An example of such a mobile digital fluoroscopy system is described in patent application WO 03/077762.

Further examples of related art are shown in the following publications:

U.S. Pat. No. 6,789,941

U.S. Pat. No. 7,231,014

U.S. Pat. No. 6,431,751

US2C212308A1

US2C213338A1

US20070255292

U.S. Pat. No. 7,403,591

### SUMMARY OF THE INVENTION

The general object of the invention is to provide improvements in a digital fluoroscopy system for medical applications operating with an X-ray device mounted to generate X-ray images, in particular such a system operating with one X-ray device mounted on a G-arm to generate X-ray images in mutually intersecting planes. Embodiments of the invention provide such improvements, as described herein.

The digital fluoroscopy system comprising a G-arm may also be referred to as a G-arm system, or a G-stand.

Further more specific objects relate to the following partial problems.
1. Weight of the G-arm system.
2. Space requirements of the G-arm system in long distance transport.
3. Space requirement in transport within building.
4. Eliminate movable parts.
5. Space within the G-arm.
6. Operability in G-arm rotational displacement.
7. Tilting displacement of G-arm.

Embodiments presented herein solve, or provide improvements with relation to, any or all of the stated partial problems.

The object is fulfilled and the partial problems are solved by embodiments of the invention as described below and in the accompanying claims.

Embodiments of the invention comprise a mobile G-arm fluoroscopy system provided with flat digital X-ray detectors. According to embodiments, it would also be possible to use flat X-ray detectors that are not digital.

According to an embodiment, there is provided a mobile digital fluoroscopy system, comprising a mobile unit 1 having a stand having a G-arm 18 suspended on a chassis frame 7; a first X-ray device 19 mounted on the G-arm 18 to generate X-ray images in a first plane P1, the first X-ray device 19 having a first receiver 22 mounted on the G-arm 18 and a first transmitter 21 mounted on the G-arm 18 opposite said first receiver 22; a second X-ray device 20 mounted on the G-arm 18 to generate X-ray images in a second plane P2 intersecting the first plane P1 of the first X-ray device, the second X-ray device 20 having a second receiver 24 mounted on the G-arm 18 and a second transmitter 23 mounted on the arm 18 opposite said second receiver 24, wherein said first and second receivers 22 and 24 are flat digital X-ray detectors mounted at respective ends of the G-arm.

In an embodiment, the flat detectors are mounted at the respective ends 104 of the G-arm with a mounting element 102 that couples the detectors to the G-arm.

In an embodiment, the mounting is configured such that the flat detectors are positioned as an extension of the G and within the outer contour of said extension of the G-arm.

In an embodiment, the mounting is configured such that it is fixed and provides a fixed non-displaceable mounting of the detector to the G-arm.

In an embodiment, the mounting of the flat detectors is configured such that the X-ray receiving surfaces of the flat detectors are positioned dose to the inner contour of said extension of the G-arm.

In an embodiment, the system comprises a balance weight 106 positioned close to each of the flat detectors, e.g. behind the detector or e.g. mounted to or integrated in or with the mounting element 102.

In an embodiment, the balance weights are selected and positioned such that the G-arm is statically balanced with regard to rotation about the rotational axis.

In an embodiment, the G-arm is made in one piece with a recess for mounting and integrating the detector in the respective end parts of the G-arm and shaped such that the G-arm with detectors mounted is statically balanced.

In an embodiment, the system comprises a suspension of the G-arm that enables a tilting or pivoting displacement of the G-arm about a horizontal axis.

In an embodiment, the weight of the G-arm components is adjusted to the weight of the chassis such that that the chassis frame balances the G-arm when tilted.

### BRIEF DESCRIPTION OF THE DRAWINGS

The present invention will be further explained below with reference to the accompanying drawings, in which:

FIG. 1-FIG. 8 show a schematic overview of an embodiment of the invention in a digital fluoroscopy system configured on a G-arm, wherein

FIG. 1 shows a perspective view of the system seen from a first direction;

FIG. 2 shows a perspective view of the system seen from a second direction;

FIG. 3 shows the system in a first side elevation;

FIG. 4 shows the system from a second side elevation;

FIG. 5 shows the system from a rear elevation;

FIG. 6 shows the system from a front elevation;

FIG. 7 shows the system from a top elevation;

FIG. 8 shows the system from a bottom elevation;

FIG. 9 shows a schematic view of details of the system according to an embodiment;

FIG. 10 shows a schematic view of a fluoroscope system comprising a mobile unit and a control unit;

FIGS. 11-13 show schematic views of details of the system according to embodiments.

### DETAILED DESCRIPTION OF THE INVENTION

### System Overview

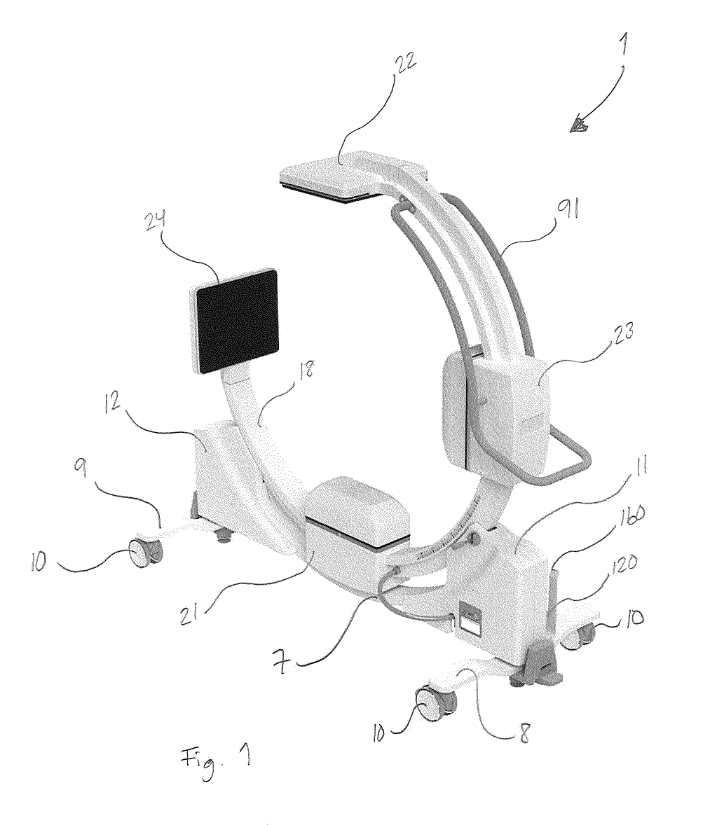

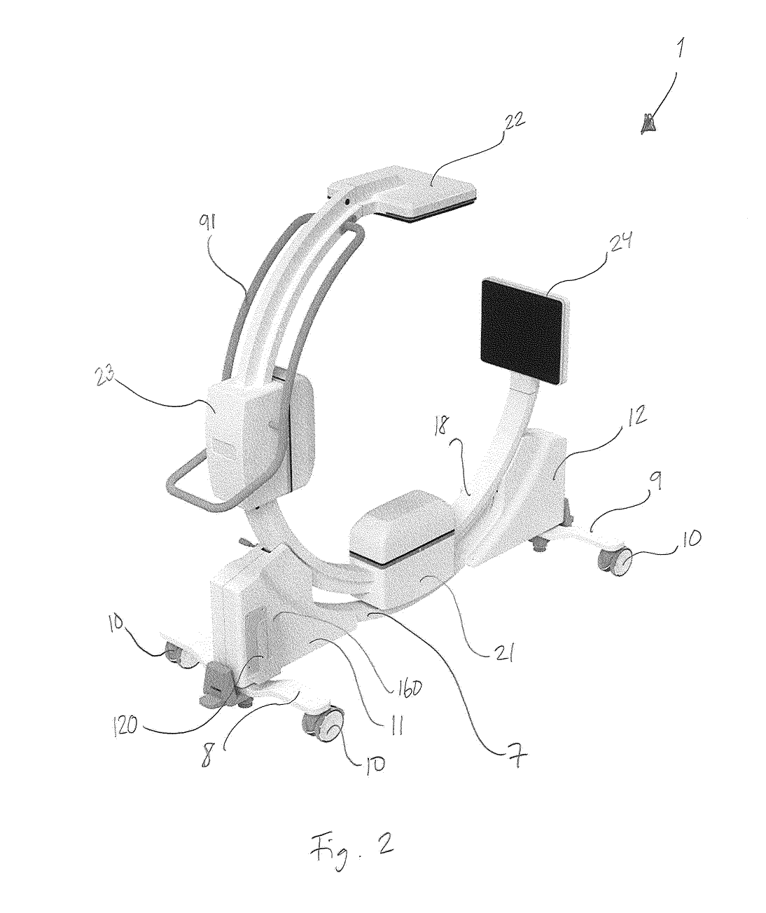

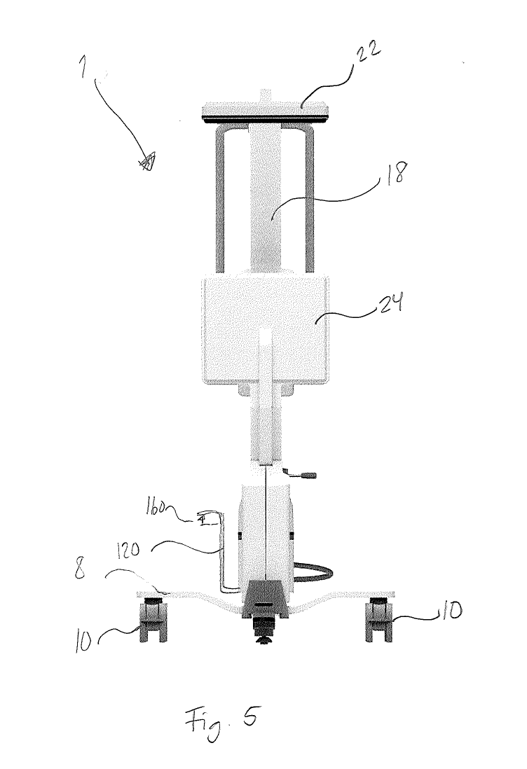

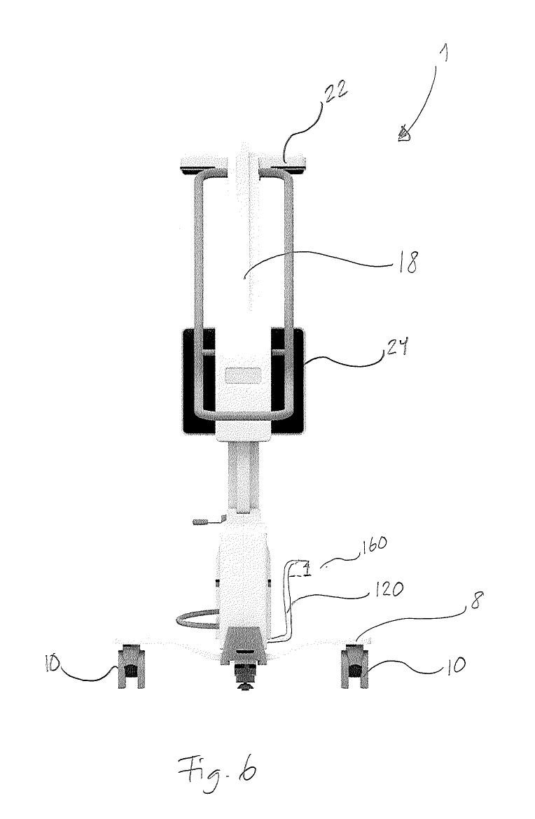

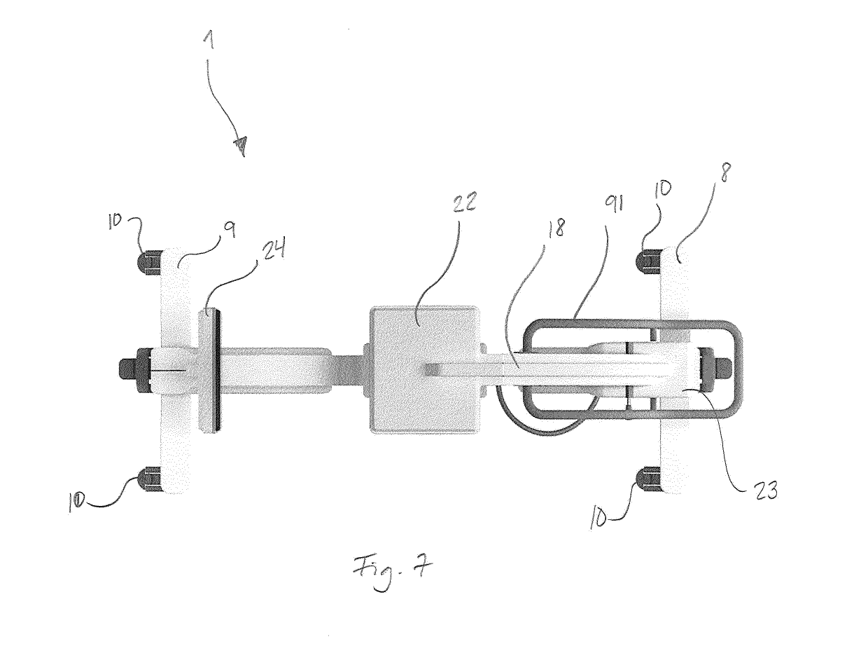

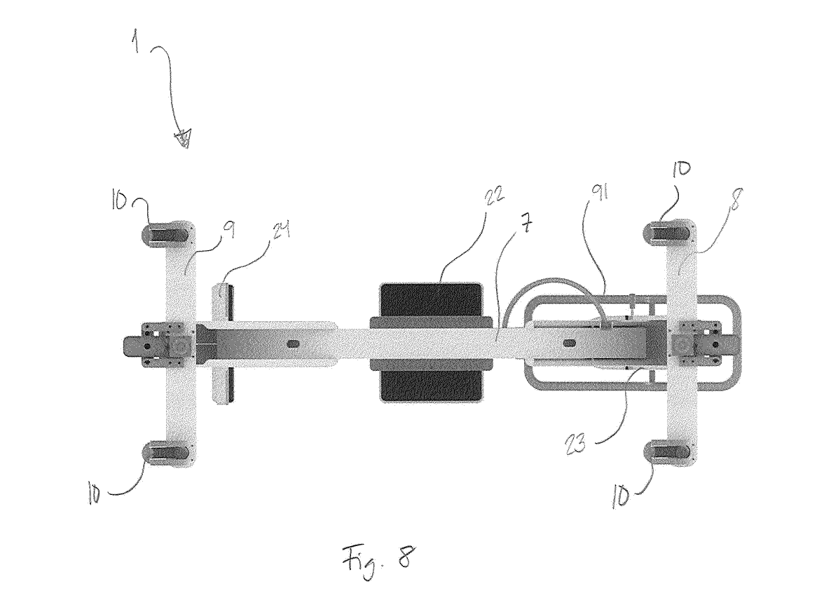

The present invention concerns an X-ray apparatus configured as a system of components illustrated in FIG. 1 to FIG. 8, adapted for use in connection with surgical orthopedic operations.

The apparatus shown in FIG. 1 to FIG. 8 comprises a mobile unit 1 provided with two X-ray systems 19, 20 mounted to operate and generate X-ray images in mutually intersecting planes P1, P2. The arm 18 of the embodiment illustrated in FIG. 1 is referred to as a G-arm.

An object, typically the body of a patient undergoing surgery, is placed inside the mobile unit 1 so that plane P1 and plane P2 of the two X-ray systems crosses the object. The first X-ray device 19 includes a first transmitter 21 (an X-ray tube or x-tube) for emitting X-rays and a first receiver 22 (e.g. image intensifier or semiconductor sensors) for receiving X-rays emitted by the first transmitter 21 and having passed through an object. The first transmitter 21 may be located down below on the arm 18 and the first receiver 22 at the top of the arm 18.

The second X-ray device 20 includes a second transmitter 23 (an X-ray tube or x-tube) for emitting X-rays and a second receiver 24 (e.g. image intensifier or semiconductor sensors) for receiving X-rays emitted by the second transmitter 23 and having passed through said object. The receivers 22, 24 may each comprise image intensifying means and an image capturing device, typically a CCD camera, for converting X-rays into a visible image.

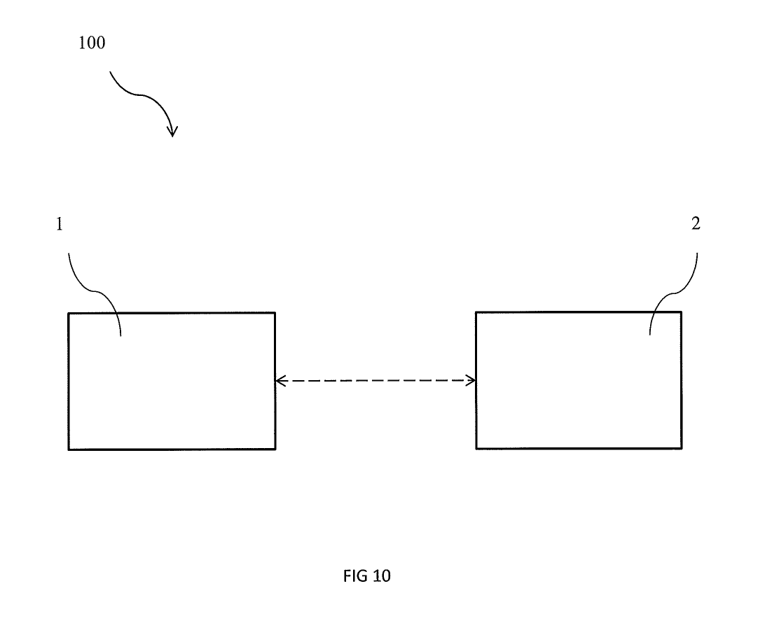

The system may further also comprise components such as a not shown foot switch for alternating between images taken in the respective planes, and also not shown high resolution monitors for presenting images to a user. The system further typically comprises a control unit comprising at least one display for displaying image data, a control panel, and a data processor comprising image processing means adapted to receive images transmitted from said image capturing devices comprised in said receivers 22, 24. This is illustrated in the schematic view in FIG. 10, wherein a system 100 comprises a mobile unit 1 and a control unit 2. The mobile unit 1 and the control unit 2 are communicatively coupled to each other, for instance by means of a cable or through wireless signal transmission, which is indicated by the dashed arrow in FIG. 10.

Generally, in FIGS. 1-13, the following reference numbers refer to the listed parts of the fluoroscopy system, wherein any or all of the listed parts may be included according to different embodiments described herein:

1 Mobile unit

2 Control unit

7 Chassis frame

8, 9 Wheel units

10 Wheels

11, 12 Vertical columns, allowing vertical adjustments

18 G-arm

19 First X-ray device

20 Second X-ray device

21 First transmitter

22 First receiver

23 Second transmitter

24 Second receiver

P1, P2 Intersecting planes

91 Handle

100 Fluoroscope system, or fluoroscopy system

120 Foot pedal unit holder

160 Cable holder

102 Mounting element

102a First part of mounting element

102b Second part of mounting element

104 Respective ends of the G-arm

106 Balance weight

1101 Linear extension of the G-arm

1102 Linear extension of the G-arm

1103 Intersection point between 1101 and 1102

### Embodiments and Features of the Invention

### Flat Detectors

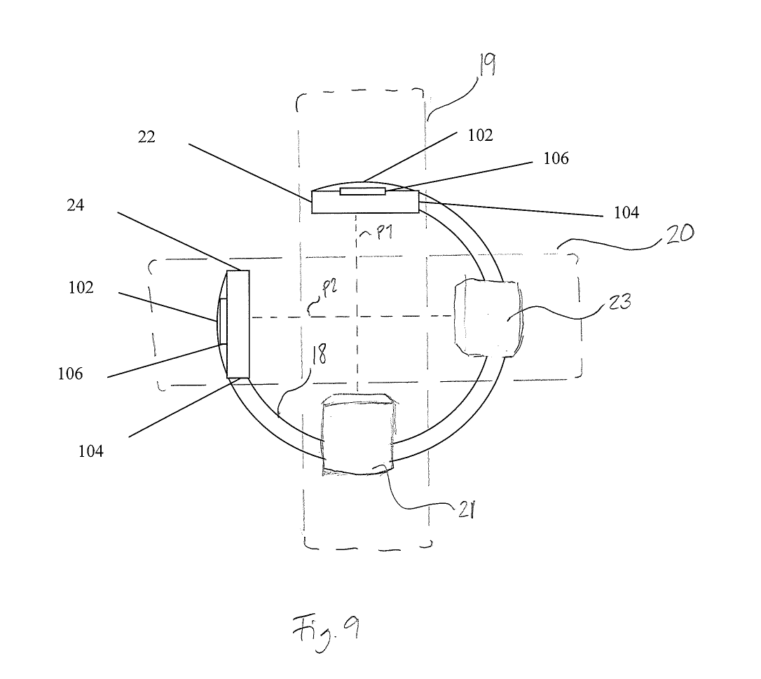

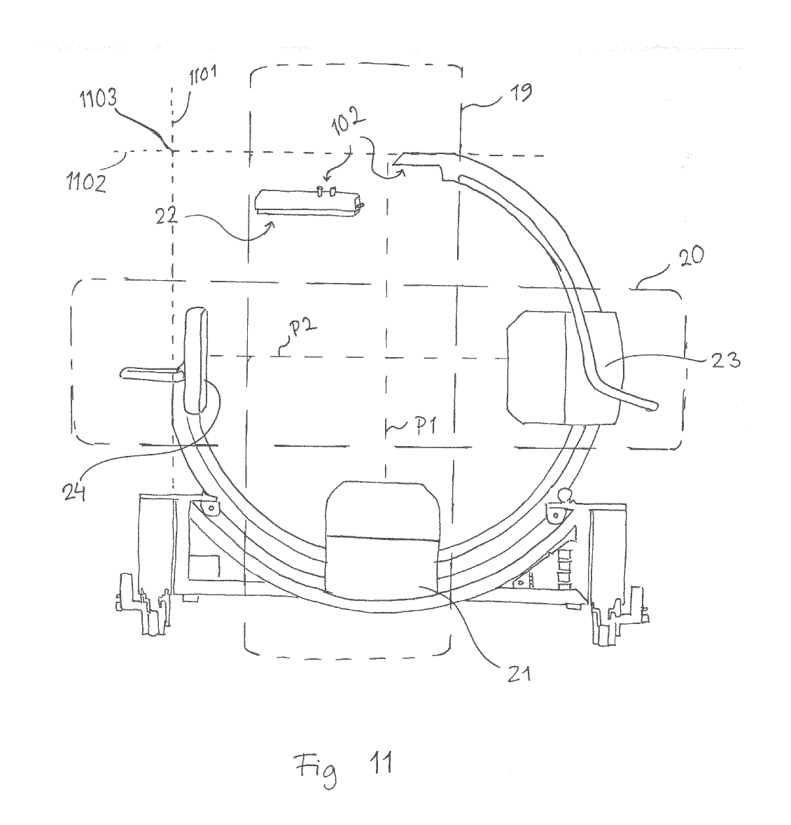

According to an embodiment of the invention schematically illustrated in FIG. 9, the flat detectors are mounted at the respective ends 104 of the G-arm with a mounting element 102 that couples the detectors to the G-arm. FIGS. 11-13 show schematic views of details of the system including a mounting element 102 according to another embodiment. Further examples of mounting elements according to different embodiments are given below.

In embodiments of the inventive concept, the mounting element 102 is a part or extension of the respective end 104 of the G-arm 18 and adapted to incorporate, or be attached or mechanically coupled to, the respective flat detectors, or receivers 22, 24. Each mounting element 102 may include elements adapted to be attached to an existing part of a receiver 22, 24 through the design of the element or using any kind of mechanical coupling, adhesive material, and/or elements adapted to attach to, be mechanically coupled to or enclose all or parts of a receiver 22, 24. In these embodiments, no adaptation of the receivers 22, 24 is required in order to enable mounting on the G-arm 18.

In embodiments of the inventive concept, the mounting element 102 is a part or extension of the respective flat detectors, or receivers 22, 24, and adapted to be attached or mechanically coupled to, the respective end 104 of the G-arm 18. Each mounting element 102 may include elements adapted to be attached to an existing part of the respective end 104 of the G-arm 18 through the design of the element or using any kind of mechanical coupling, adhesive material, and/or elements adapted to enclose all or parts of the respective end 104 of the G-arm 18. In these embodiments, no adaptation of the G-arm 18 is required in order to enable mounting of the receivers 22, 24.

In other embodiments, the mounting element 102 is a separate module adapted to be attached or mechanically coupled to, or to incorporate, one of the flat detectors, or receivers 22, 24, and further adapted to be attached or mechanically coupled to the respective end 104 of the G-arm 18. Each mounting element 102 may include elements adapted to be attached to an existing part of the respective end 104 of the G-arm 18, and/or receivers 22, 24, through the design of the element or using any kind of mechanical coupling, adhesive material, and/or elements adapted to enclose all or parts of the respective end 104 of the G-arm 18 and/or receivers 22, 24. In these embodiments, no adaptation of the G-arm is required in order to enable mounting of the receivers 22, 24.

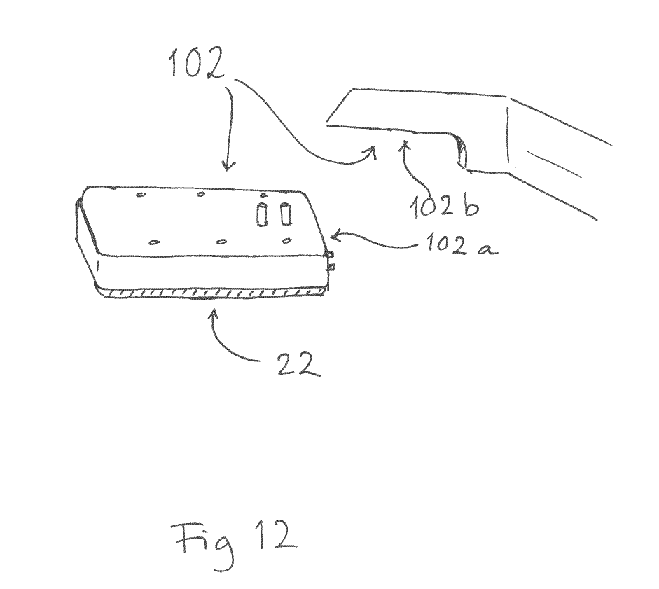

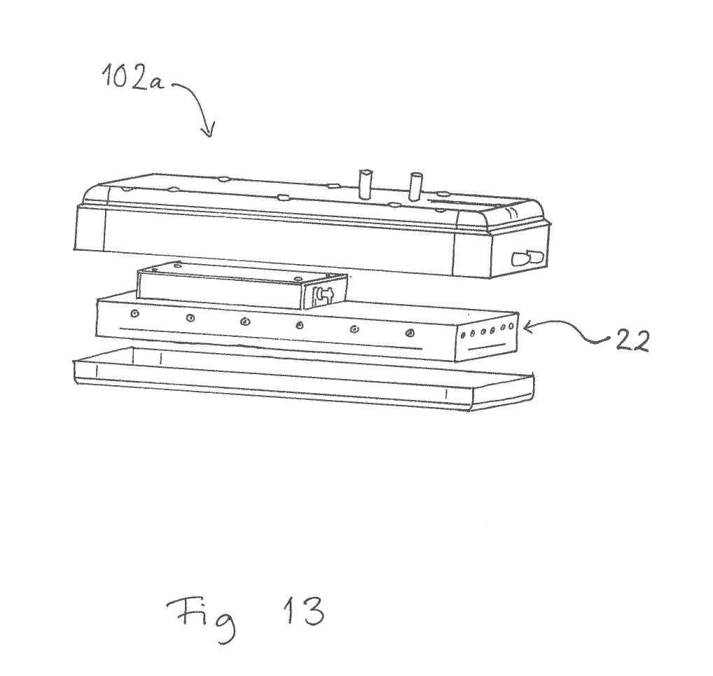

In other embodiments, the mounting element 102 consists of a first part iota and a second part 132b that are adapted to be attached or mechanically coupled to each other, wherein the first part iota of the mounting element 102 is further adapted to be attached to, mechanically coupled to, or incorporate, one of the flat detectors, or receivers 22, 24 and the second part limb of the mounting element 102 is further adapted to be attached or mechanically coupled to the respective end 104 of the G-arm 18. The first part iota of the mounting element 102 may according to embodiments comprise one or more elements adapted to be attached or mechanically coupled to an existing part of the receivers 22, 24, through the design of the element or using any kind of adhesive material, and/or elements adapted to enclose all or parts of the receivers 22, 24. The second part limb of the mounting element 102 may according to embodiments comprise one or more elements adapted to be attached or mechanically coupled to an existing part of the G-arm 18. Each mounting element 102, or mounting element part 102a, limb, may include elements adapted to be attached to an existing part of the respective end 104 of the G-arm 18 through the design of the element, or element part, or using any kind of adhesive material, mechanical coupling and/or elements adapted to enclose all or parts of the respective end 104 of the G-arm 18. An example, according to one of many possible embodiments, of a mounting element 102 having a first part iota and a second part 102b that comprise mating or matching mechanical coupling elements is shown in FIGS. 11-12. In FIG. 13 an example of a first part iota of the mounting element 102 is shown, wherein the first part iota is a box that encloses one of the receivers, the receiver 22. The box may further be coupled to a second part 102b of the mounting element 102, as exemplified in FIG. 12.

The mounting element 102, according to any of the embodiments described herein, may be designed according to any known method of fastening, attaching, or incorporating modules that would enable the respective flat detectors, or receivers 22, 24, to be attached or mechanically coupled to the respective ends 104 of the G-arm 18.

Preferably, the mounting element 102 is configured such that it provides a fixed non-displaceable mounting of the detectors 22,24 to the G-arm 18, or more specifically, such that the detectors 22, 24 are fixed, non-displaceable, in relation to the G-arm 18 after assembly of the system.

The flat detectors reduce weight of the G-arm system. According to the embodiments described herein, the flat detectors are digital detectors. However, it would also be possible to use flat X-ray detectors that are not digital.

Detector within Outer Contour of G-arm

The mounting is configured such that the flat detectors are positioned as an extension of the G and within the outer contour of said extension of the G-arm. This has the effect that the space requirement of the G-arm system is reduced.

This is important for example in long distance transport for this kind of apparatus, which is generally large. In transport, the space allowed for goods is limited and confined to standard size boxes. This is a problem for prior art apparatus with X-ray detectors that extend outside and beyond the outer G-arm contour. Prior art apparatus usually require disassembling.

As seen in FIG. 9, the extension of the G-arm 18 may have an outer contour that is a continuation of the outer perimeter of the G-arm arc, whereby the extension of the G-arm 18 is consequently also a continuation of the arc; i.e. the extension of the G-arm 18 is in the shape of a circle segment that connects the two ends of the G-arm 18. As can be seen from FIG. 9, the flat detectors, or receiver 22, 24, may according to embodiments be mounted such that the receivers 22, 24 are positioned more or less within the inner space of G-arm 18. For example, the receivers 22, 24 may be fastened at or near the respective ends 104 of the G-arm 18 using a mounting element 102 that positions the receiver 22, 24 more or less within the inner space of the G-arm 18. In embodiments, the receivers 22, 24 stand out from the respective end 104 of the G-arm 18 when they are mounted on the G-arm 18. In embodiments, the receiver 22 could be seen as “hanging” from the respective end 104 of the G-arm 18 when it is mounted on the G-arm 18.

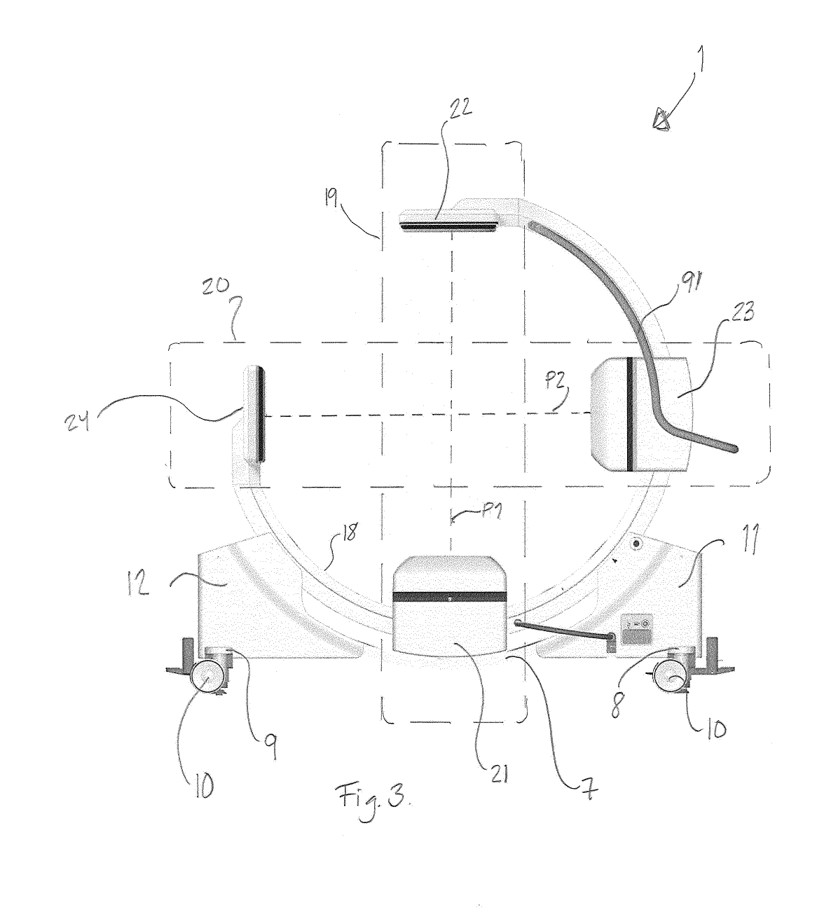

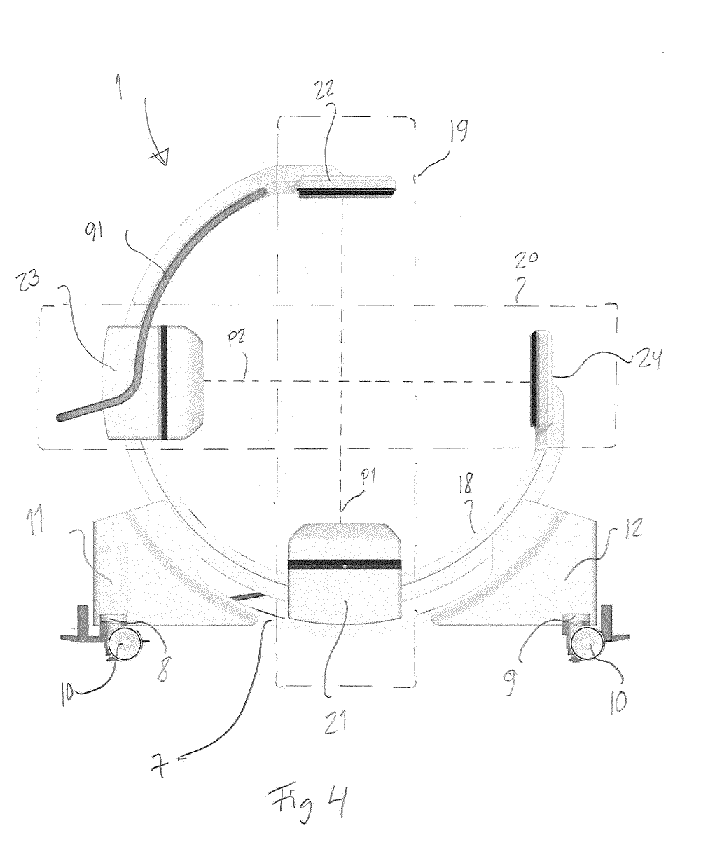

As seen in FIGS. 3-4 and ii, the extension of the G-arm 18 may have an outer contour that is linear and in normal use is typically perpendicular to the plane P1 for the end where the receiver 22 is coupled to the G-arm 18, or parallel to the plane P1 for the end where the receiver 24 is coupled to the G-arm 18. According to this embodiment, the outer contour of the extension of the G-arm consequently consists of two linear extensions that intersect. In FIG. 11, exemplary linear extensions 1101, 1102 are indicated with dotted lines having an intersection point 1103.

Both the circle segment extension shape and the linear extension shape provide the advantage of reducing the space requirement. Furthermore, both embodiments are equivalently beneficial for allowing the G-arm to fit within rectangular or rectangular block boundaries, for example standard boxes for shipping or hospital doors, as discussed herein.

### Fixed Mounting of Detectors

The mounting element 102 is further preferably configured such that it is firm or fixed, i.e. that it provides a fixed non-displaceable mounting of the detector to the G-arm. This has the effect to eliminate the need for a displaceable mounting element. The mounting element 102 may be of any of the types described herein.

This is important in transport within buildings on the wheels of the apparatus, typically hospital buildings where this kind of apparatus is in use. Doors and ceiling height have dimensions that for prior art apparatus require that the detectors, particularly the top detector, is dismounted or displaced. Prior art apparatus have a displaceable detector with a displaceable mounting arrangement to the G-arm. The need for this displaceable element is eliminated by the solution according to the invention. The invention also simplifies and makes more efficient the work in hospitals since the need for adjusting the top detector out of operating settings for moving the apparatus is eliminated.

### Detector Along Inner Contour of G-arm

The mounting of the flat detectors is further preferably configured such that the X-ray receiving surfaces of the flat detectors are positioned close to the inner contour of said extension of the G-arm. In other words, the X-ray receiving surfaces of the flat detectors are positioned along the inner contour or perimeter of the G-arm. In yet other words, the flat detectors, or receivers 22, 24, do not take up any significant space within the G-arm. This has the effect that the space within the G-arm is enlarged. In use, a patient on an operation table, a surgeon, various surgery staff and various equipment will be positioned within the G-arm. The flat detectors and particularly the mounting close to the inner contour increase the available space and the accessibility to the patient within the G-arm significantly.

As seen in FIG. 9, the extension of the G-arm 18 may have an inner contour or perimeter that is a continuation of the inner border of the G-arm arc, whereby the extension of the G-arm is consequently also a continuation of the arc; i.e. the extension of the G-arm is in the shape of a circle segment that connects the two ends of the G-arm.

As seen in FIGS. 3-4 and 11, the extension of the G-arm may have an inner contour that is linear and in normal use is typically perpendicular to the plane P1 for the end where the receiver 22 is coupled to the G-arm 18, or parallel to the plane P1 for the end where the receiver 24 is coupled to the G-arm 18. According to this embodiment, the inner contour of the extension of the respective ends 104 of the G-arm consequently consists of two linear extensions that intersect.

Both the circle segment extension shape and the linear extension shape provide the advantage of reducing the space requirement. Furthermore, both embodiments are equivalently beneficial for allowing the G-arm to fit within rectangular or rectangular block boundaries, for example standard boxes for shipping or hospital doors, as discussed herein.

According to embodiments, the mounting of the flat detectors are configured such that they are positioned as an extension of the G-arm within the outer contour of the extension of the G-arm, and wherein the X-ray receiving surfaces of the detectors are at the same time positioned close to the inner contour of the extension of the G-arm. Thereby, the combined advantages of reducing the space requirement of the G-arm system and enlarging, or maximizing, the space within the G-arm are achieved.

### Fixed Mounting and Balanced G-arm

In one embodiment, the system comprises a balance weight 106 (Cf. FIG. 9) positioned close to each of the flat detectors, e.g. behind the detector or e.g. mounted to or integrated in or with the mounting element 102. The G-arm is substantially a ¾ circular arc that is supported on a chassis frame such that the G-arm can rotate about an axis through the center of the circular arc and thereby the position of the X-ray devices be adjusted. According to this embodiment of the invention, the balance weights are selected and positioned such that the G-arm is statically balanced with regard to rotation about the rotational axis. In order to adjust the rotational position of the G-arm, an operator only has to apply a small force to set the G-arm in motion, find the desired position and lock the G-arm with a locking mechanism also provided in the system. This solution has inter alia the effects that the operability of the G-arm is improved, the risk for squeeze injuries on operators is reduced and mechanisms for driving the adjustment motion of the G-arm as well as mechanisms for keeping the arm in position are reduced.

In another embodiment, the G-arm is made in one piece with a recess for mounting and integrating the detector in the respective end parts of the G-arm and shaped such that the G-arm with detectors mounted is statically balanced.

### Tilting Displacement of G-arm

Embodiments of the invention comprise a suspension of the G-arm that enables a tilting or pivoting displacement of the G-arm about a horizontal axis. This is also enabled by the flat detector giving the G-arm components a sufficiently low weight that is adjusted to the weight of the chassis such that that the chassis frame balances the G-arm when tilted.

1) A mobile digital fluoroscopy system, comprising a mobile unit (1) having:
a stand having a G-arm (18) suspended on a chassis frame (7);
a first X-ray device (19) mounted on the G-arm (18) to generate X-ray images in a first plane (P1), the first X-ray device (19) having a first receiver (22) mounted on the G-arm (18) and a first transmitter (21) mounted on the G-arm (18) opposite said first receiver (22);
a second X-ray device (20) mounted on the G-arm (18) to generate X-ray images in a second plane (P2) intersecting the first plane (P1) of the first X-ray device, the second X-ray device (20) having a second receiver (24) mounted on the G-arm (18) and a second transmitter (23) mounted on the arm (18) opposite said second receiver (24),
wherein said first and second receivers (22) and (24) are flat digital X-ray detectors mounted at respective ends of the G-arm.

2) The system of claim 1, wherein said flat detectors are mounted at the respective ends (104) of the G-arm with a mounting element (102) that couples the detectors to the G-arm.

3) The system of claim 1, wherein the mounting is configured such that the flat detectors are positioned as extensions of the G-arm and within the outer contour of said extension of the G-arm.

4) The system of claim 3, wherein said extensions of the G-arm (18) are continuations of the outer perimeter of the G-arm arc.

5) The system of claim 3, wherein said extensions of the G-arm (18) are in the shape of circle segments that connect the two ends of the G-arm (18).

6) The system of claim 3, wherein said extension of the G-arm (18) are linear.

7) The system of claim 6, wherein said extensions of the respective ends (104) of the G-arm (18) consists of two linear extensions, wherein the longitudinal axes of said two linear extensions intersect.

8) The system of claim 1, wherein the mounting is configured such that it is fixed and provides a fixed non-displaceable mounting of the detector to the G-arm.

9) The system of claim 3, wherein the mounting of each flat detector is configured such that its X-ray receiving surfaces of each flat detector is positioned close to the inner contour of its respective extension of the G-arm.

10) The system of claim 1, wherein the system comprises a balance weight 106 positioned close to each of the flat detectors, e.g. behind the detector or e.g. mounted to or integrated in or with the mounting element (102).

11) The system of claim 10, wherein the balance weights are selected and positioned such that the G-arm is statically balanced with regard to rotation about the rotational axis.

12) The system of claim 1, wherein the G-arm is made in one piece with a recess for mounting and integrating the detector in its respective end the G-arm and is shaped such that the G-arm with detectors mounted is statically balanced.

13) The system of claim 1, wherein the system comprises a suspension of the G-arm that enables a tilting or pivoting displacement of the G-arm about a horizontal axis.

14) The system of claim 1, wherein the weight of the G-arm components is adjusted to the weight of the chassis such that that the chassis frame balances the G-arm when tilted.

15) A mobile digital fluoroscopy system, comprising a mobile unit (1) having:
a stand having a G-arm (18) suspended on a chassis frame (7);
a first X-ray device (19) mounted on the G-arm (18) to generate X-ray images in a first plane (P1), the first X-ray device (19) having a first receiver (22) mounted on the G-arm (18) and a first transmitter (21) mounted on the G-arm (18) opposite said first receiver (22);
a second X-ray device (20) mounted on the G-arm (18) to generate X-ray images in a second plane (P2) intersecting the first plane (P1) of the first X-ray device, the second X-ray device (20) having a second receiver (24) mounted on the G-arm (18) and a second transmitter (23) mounted on the arm (18) opposite said second receiver (24),
wherein said first and second receivers (22) and (24) are flat digital X-ray detectors mounted at respective ends of the G-arm;
wherein the mounting is configured such that the flat detectors are positioned as extensions of the G-arm and within the outer contour of said extensions of the G-arm.

16) The system of claim 15, wherein each flat detectors is mounted at respective end (104) of the G-arm with a respective mounting element (102) that couples the detectors to the G-arm.

17) The system of claim 15, wherein said extensions of the G-arm (18) are continuations of the outer perimeter of the G-arm arc.

18) The system of claim 15, wherein said extensions of the G-arm (18) are in the shape of a circle segment that connects the two ends of the G-arm (18).

19) The system of claim 15, wherein said extensions of the G-arm (18) are linear.

20) The system of claim 15, wherein said extensions of the G-arm (18) of each the respective end (104) of the G-arm (18) consist of two linear extensions, wherein the longitudinal axes of said two linear extensions intersect.

21) The system of claim 15, wherein the mounting is configured such that it is fixed and provides a fixed non-displaceable mounting of each detector to the G-arm.

22) The system of claim 15, wherein the mounting of the flat detectors is configured such that the X-ray receiving surfaces of the flat detectors are positioned close to the inner contour of said extensions of the G-arm.

23) The system of claim 15, wherein the system comprises a balance weight 106 positioned close to each of the flat detectors, e.g. behind the detector or e.g. mounted to or integrated in or with the mounting element (102).

24) The system of claim 15, wherein the balance weights are selected and positioned such that the G-arm is statically balanced with regard to rotation about the rotational axis.

25) The system of claim 15, wherein the G-arm is made in one piece with a recess for mounting and integrating the detector in the respective end of the G-arm and shaped such that the G-arm with detectors mounted is statically balanced.

26) The system of claim 15, wherein the system comprises a suspension of the G-arm that enables a tilting or pivoting displacement of the G-arm about a horizontal axis.

27) The system of claim 15, wherein the weight of the G-arm components is adjusted to the weight of the chassis such that that the chassis frame balances the G-arm when tilted.
